(19) United States
(12) Patent Application Publication
(10) Pub. No.: US 2024/0226046 A1
(43) Pub. Date: Jul. 11, 2024

(54) COMPOSITION CONTAINING LYSINE FOR THE TREATMENT OF POISON IVY, COMPOSITION CONTAINING LYSINE FOR THE PREVENTION OF POISON IVY, PROCESSES, AND METHOD OF USE

(71) Applicant: Douglas J. Tataryn, Selkirk (CA)

(72) Inventor: Douglas J. Tataryn, Selkirk (CA)

(21) Appl. No.: 18/399,935

(22) Filed: Dec. 29, 2023

Related U.S. Application Data
(60) Provisional application No. 63/438,286, filed on Jan. 11, 2023.

(51) Int. Cl. A61K 31/198

(52) U.S. Cl. CPC A61K 31/198

(57) ABSTRACT

A composition containing lysine for the treatment of poison ivy and/or poison oak, a composition containing lysine for the prevention of poison ivy and/or poison oak, processes, and methods of use are presented. Thus, a composition and treatment including Lysine for the treatment and/or prevention of urushiol-induced contact dermatitis (otherwise known as poison ivy and/or poison oak) is presented. The disclosure provides a composition containing lysine for the treatment and/or prevention of effects caused by exposure to urushiol and/or a liquid composed of plant sap. More specifically, the present disclosure relates to a supplement which can be taken daily and/or as a treatment for poison ivy and/or other allergens and/or allergic reactions.

### CROSS-REFERENCE TO RELATED APPLICATIONS

The present application claims priority to the U.S. Provisional Patent Application No. 63/438,286 which was filed on Jan. 11, 2023, which is hereby incorporated by reference herein in its entirety, including any figures, tables, or drawings.

### FIELD OF THE DISCLOSURE

This disclosure relates to a composition and treatment including Lysine for the treatment and/or prevention of urushiol-induced contact dermatitis (otherwise known as poison ivy and/or poison oak). More specifically, and without limitation, the present disclosure relates to a composition containing lysine for the treatment and/or prevention of effects caused by exposure to urushiol and/or a liquid composed of plant sap. More specifically, and without limitation, the present disclosure relates to a supplement which can be taken daily and/or as a treatment for poison ivy and/or other allergens and/or allergic reactions.

### COPYRIGHT NOTICE

At least a portion of the disclosure of this patent document contains material that is subject to copyright protection. The copyright owner has no objection to the facsimile reproduction by anyone of the patent document or the patent disclosure, as it appears in the Patent and Trademark Office patent files and/or records, but otherwise reserves all copyright rights whatsoever. The following notice applies to the software and data as described below and in the drawings that form a part of this document. Copyright. Dr. Douglas J. Tataryn. All rights reserved.

### BACKGROUND OF THE DISCLOSURE

Poison ivy is old and well known in the art. Poison ivy is a common name for a both—a type of reaction and/or side effect to an exposure to a plant, as well as, a type of plant and/or group of plant species.

More specifically, poison ivy as a condition, refers to skin irritation as an allergic reaction to urushiol. As a condition, this exposure can result in a reaction which is a painful rash for most people who are exposed. This rash is itchy and can be very extreme in some cases and almost always very uncomfortable for the person experiencing the condition. While some of the population has no reaction, the condition affects a majority of the population. At a minimum, discomfort, itchiness, rash, and blisters are common. Most of the time, extreme discomfort and allergic reactions are much more intense. Furthermore, the oil can spread from human to human through contact with one another or through objects which are touched as the oil spreads.

More specifically, poison ivy as a plant releases a liquid compound, or plant sap, which contains urushiol. Urushiol, the compound, is what causes irritation and allergic reactions for many who are exposed. This plant is a common weed which is easily spread and common in various parts of the world. Hidden as small plants, shrubs, and in some cases vines, the plants generally have clusters of leaves which carry sap and can cause poison ivy, often to unsuspecting hikers or others experiencing the outdoors.

There are no known treatments for the treatment and/or prevention of poison ivy. While recommendations include scrubbing the affected area with soap, potentially applying lotions or potentially certain steroids (which may help mitigate symptoms), treatments such as itch creams and/or washing are only effective if caught immediately for washing or to provide some relief from symptoms. Thus, there is a long-felt need in the art for a composition which is effective in treating poison ivy. Furthermore, there is a long-felt need in the art for a composition which is effective at preventing poison ivy reactions.

Lysine is well known in the art. Lysine is an essential amino acid. While the human body is not known to make lysine, lysine is an important component in the successful making of proteins in the human body. Lysine can be found in, and consumed by humans, in meat, fish, dairy, and eggs. So, lysine must be consumed in the diet to enter the body and aid as an essential building block for making proteins.

Lysine is known for some health benefits and particularly herpes. In other words, some studies have shown that taking lysine by mouth may help prevent and heal cold sores. However, any other known uses of lysine for health benefits or for medical purposes are unknown.

Thus, there is little known about lysine and little to no research into lysine as it is known as an essential amino acid. Therefore, the state of the art has a long-felt need for a poison ivy prevention and treatment while teaching away from using lysine for anything but the purpose of herpes treatment.

The disclosure herein provides these advantages and others as will become clear from the specification and claims provided.

### SUMMARY OF THE DISCLOSURE

A composition containing lysine for the treatment of poison ivy, a composition containing lysine for the prevention of poison ivy, processes, and methods of use are presented. Said another way, the present disclosure provides a composition and treatment including Lysine for the treatment and/or prevention of urushiol-induced contact dermatitis (otherwise known as poison ivy and/or poison oak). The present disclosure relates to a composition containing lysine for the treatment and/or prevention of effects caused by exposure to urushiol and/or a liquid composed of plant sap. Furthermore, the present disclosure relates to a supplement which can be taken daily and/or as a treatment for poison ivy and/or other allergens and/or allergic reactions.

Thus, it is a primary object of the disclosure to provide a composition containing lysine for the treatment of poison ivy, a composition containing lysine for the prevention of poison ivy, processes, and methods of use.

Another object of the disclosure is to provide a composition containing lysine for the treatment of poison ivy and/or poison oak, a composition containing lysine for the prevention of poison ivy and/or poison oak, processes, and methods of use that prevents reactions to poison ivy such as rashes, bumps, blisters, oozing, itching, the the like.

Yet another object of the disclosure is to provide a composition containing lysine for the treatment of poison ivy, a composition containing lysine for the prevention of poison ivy, processes, and methods of use that treats auto-immunological level conditions.

Another object of the disclosure is to provide a composition containing lysine for the treatment of poison ivy and/or poison oak, a composition containing lysine for the prevention of poison ivy and/or poison oak, processes, and methods of use that treats allergic reactions.

Yet another object of the disclosure is to provide a composition containing lysine for the treatment of poison ivy, a composition containing lysine for the prevention of poison ivy, processes, and methods of use that prevents allergic reactions.

Another object of the disclosure is to provide a composition containing lysine for the treatment of poison ivy and/or poison oak, a composition containing lysine for the prevention of poison ivy and/or poison oak, processes, and methods of use that treats allergic reactions to pollen.

Yet another object of the disclosure is to provide a composition containing lysine for the treatment of poison ivy, a composition containing lysine for the prevention of poison ivy, processes, and methods of use that prevents allergic reaction to pollen.

Another object of the disclosure is to provide a composition containing lysine for the treatment of poison ivy and/or poison oak, a composition containing lysine for the prevention of poison ivy and/or poison oak, processes, and methods of use that treats allergic reactions such as hay fever.

Yet another object of the disclosure is to provide a composition containing lysine for the treatment of poison ivy, a composition containing lysine for the prevention of poison ivy, processes, and methods of use that prevents hay fever.

Another object of the disclosure is to provide a composition containing lysine for the treatment of poison ivy and/or poison oak, a composition containing lysine for the prevention of poison ivy and/or poison oak, processes, and methods of use that prevent allergic reactions on the skin from occurring.

Yet another object of the disclosure is to provide a composition containing lysine for the treatment of poison ivy, a composition containing lysine for the prevention of poison ivy, processes, and methods of use that treats skin allergies.

Another object of the disclosure is to provide a composition containing lysine for the treatment of poison ivy and/or poison oak, a composition containing lysine for the prevention of poison ivy and/or poison oak, processes, and methods of use that treat shingles.

Yet another object of the disclosure is to provide a composition containing lysine for the treatment of poison ivy, a composition containing lysine for the prevention of poison ivy, processes, and methods of use that treat hives.

Another object of the disclosure is to provide a composition containing lysine for the treatment of poison ivy and/or poison oak, a composition containing lysine for the prevention of poison ivy and/or poison oak, processes, and methods of use that prevent bumps and/or blisters from forming on the skin.

Yet another object of the disclosure is to provide a composition containing lysine for the treatment of poison ivy, a composition containing lysine for the prevention of poison ivy, processes, and methods of use that treats bumps and/or blisters of the skin.

Another object of the disclosure is to provide a composition containing lysine for the treatment of poison ivy and/or poison oak, a composition containing lysine for the prevention of poison ivy and/or poison oak, processes, and methods of use that is easy to implement.

Yet another object of the disclosure is to provide a composition containing lysine for the treatment of poison ivy, a composition containing lysine for the prevention of poison ivy, processes, and methods of use that is safe to implement.

Another object of the disclosure is to provide a composition containing lysine for the treatment of poison ivy and/or poison oak, a composition containing lysine for the prevention of poison ivy and/or poison oak, processes, and methods of use that is quick and efficient.

Yet another object of the disclosure is to provide a composition containing lysine for the treatment of poison ivy, a composition containing lysine for the prevention of poison ivy, processes, and methods of use that improves the overall health of a user.

Another object of the disclosure is to provide a composition containing lysine for the treatment of poison ivy and/or poison oak, a composition containing lysine for the prevention of poison ivy and/or poison oak, processes, and methods of use that improves the comfort of a user.

Yet another object of the disclosure is to provide a composition containing lysine for the treatment of poison ivy, a composition containing lysine for the prevention of poison ivy, processes, and methods of use that improves recovery time to poison ivy reactions.

These and other objects, features, or advantages of the present disclosure will become apparent from the specification and claims.

### BRIEF DESCRIPTION OF THE DRAWINGS

The drawings accompanying and forming part of this specification are included to depict certain aspects of the disclosure.

FIG. 1 is an illustration depicting the overall composition of the method and the key parties involved; the illustration showing an overall method and/or system; the illustration showing a user, the user may also be considered a patient whether self-administering or being provided care; the illustration also depicting a medical professional, whether the medical professional is a doctor, nurse, nurse practitioner, or another providing care and/or assistance to the user; the illustration showing a communication system, the communication system being tied to the method, the user and/or the medical professional and/or the dose; the illustration showing a dose, the dose as applying to a user/patient, the dose being changed by the system from time to time to adjust to a user and/or the metrics of output of the communication system.

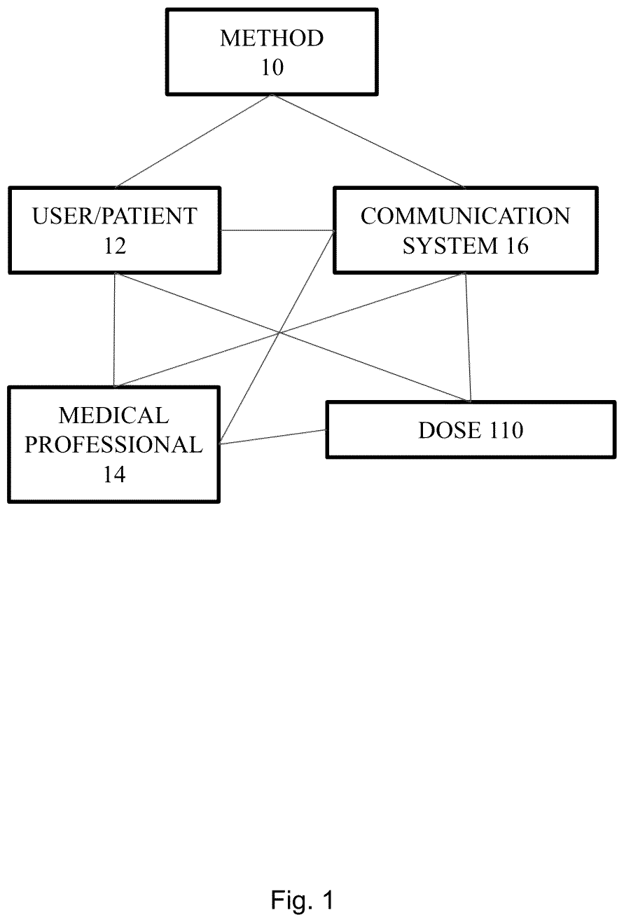

FIG. 2 is an illustration showing the dose of lysine; the illustration showing some of the various administration methods and/or possibilities of injecting the dose of l-lysine into the system of a user; the illustration showing a tablet 20 as one option of injection, the oral consumption option and ingesting l-lysine into the system of a user utilizing the digestive tract and/or gut biome of a user; the illustration showing a patch, referring to a dermal patch which injects lysine into the system of a user through transdermal application; the illustration showing a cream, referring to a cream or lotion and/or transdermal application of lysine; the illustration showing an injection method for inject lysine into the user.

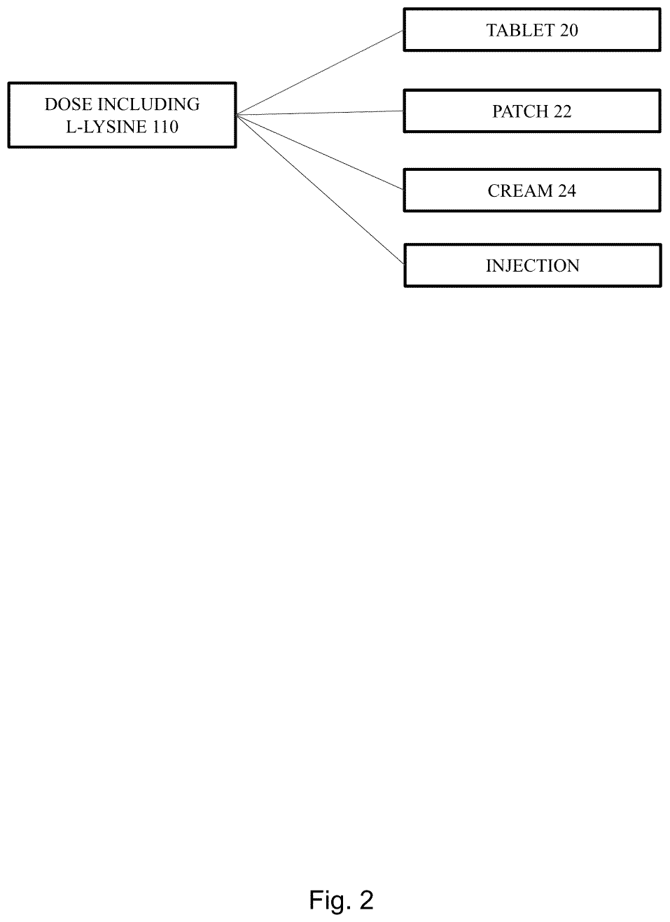

FIG. 3 is an illustration showing a dose of lysine; the illustration showing vitamins as a possible ingredient in the composition of a dose of lysine, along with a combination of other possible ingredients in the composition; the illustration showing sugars as a possible ingredient in the composition of a dose of lysine, along with a combination of other possible ingredients in the composition; the illustration showing Vitamin B1 as a possible ingredient in the composition of a dose of lysine, along with a combination of other possible ingredients in the composition; the illustration showing pill casing as a possible ingredient in the composition of a dose of lysine, along with a combination of other possible ingredients in the composition; the illustration showing Vitamin B2 as a possible ingredient in the composition of a dose of lysine, along with a combination of other possible ingredients in the composition; the illustration showing gluten as a possible ingredient in the composition of a dose of lysine, along with a combination of other possible ingredients in the composition; the illustration showing Vitamin B6 as a possible ingredient in the composition of a dose of lysine, along with a combination of other possible ingredients in the composition; the illustration showing omeprazole as a possible ingredient in the composition of a dose of lysine, along with a combination of other possible ingredients in the composition; the illustration showing Vitamin C as a possible ingredient in the composition of a dose of lysine, along with a combination of other possible ingredients in the composition; the illustration showing iron as a possible ingredient in the composition of a dose of lysine, along with a combination of other possible ingredients in the composition; the illustration showing glutamine as a possible ingredient in the composition of a dose of lysine, along with a combination of other possible ingredients in the composition.

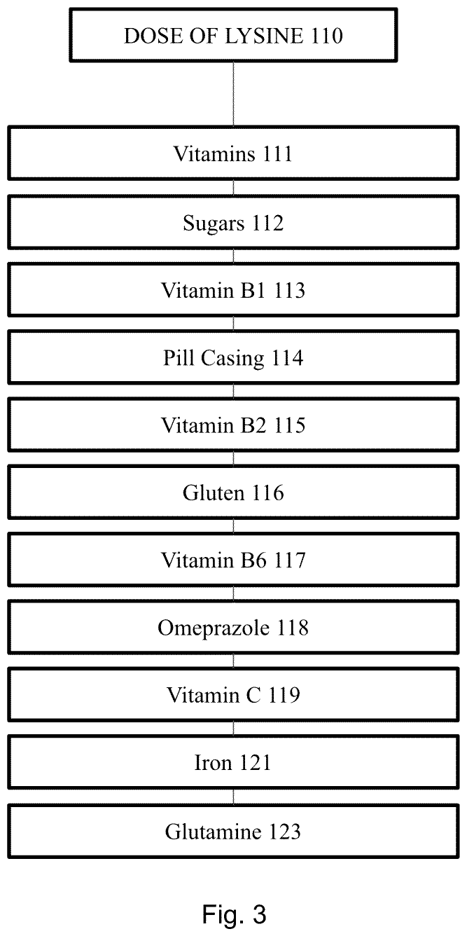

FIG. 4 is an illustration showing various absorption options which, as disclosed herein, facilitate in absorption of L-Lysine; the illustration showing absorption enhancements for those who identify as males; the illustration showing absorption enhancements for those who identify as females; the illustration showing absorption enhancements for those who identify as pregnant; the illustration showing absorption enhancements for those who identify as breastfeeding; the illustration showing absorption enhancements for Vitamin B1; the illustration showing absorption enhancements for Vitamin B2; the illustration showing absorption enhancements for Vitamin B6; the illustration showing absorption enhancements for Vitamin C; the illustration showing absorption enhancements for Glutamine.

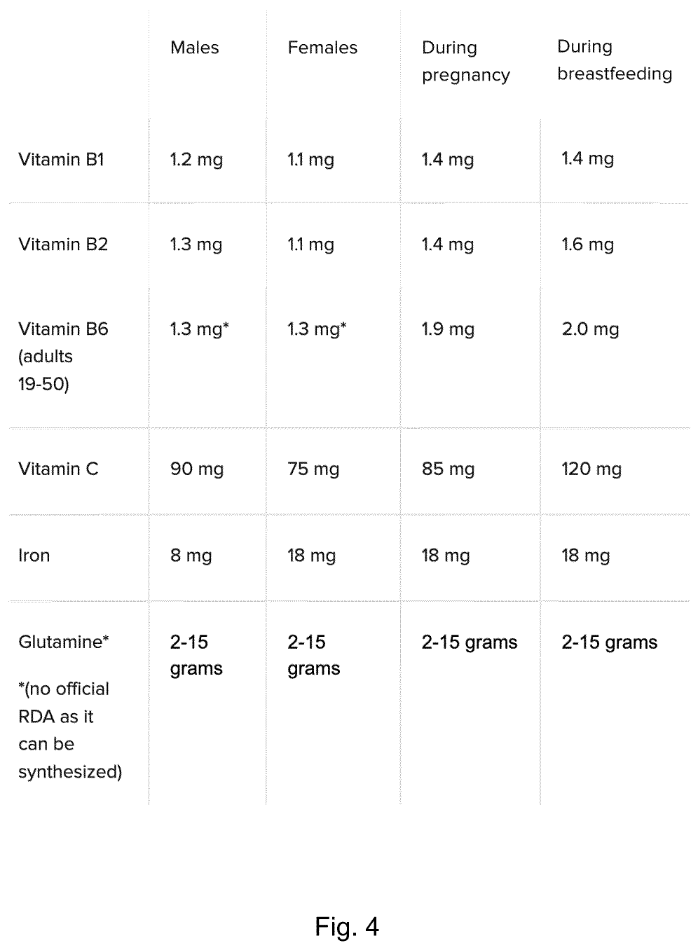

The drawings accompanying and forming part of this specification are included to depict certain aspects of the disclosure and are not intended to be limiting but intended to provide examples of certain illustrations.

It will be appreciated by those skilled in the art that other various modifications could be made to the illustrations, the compositions of these illustrations, the processes of these illustrations, the dosages illustrated, and methods of use without parting from the spirit and scope of this disclosure. All such modifications and changes fall within the scope of the claims and are intended to be covered thereby.

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

### User:

In the arrangement shown, as one example, the composition for a dose of lysine is injected into, absorbed by, ingested by, a combination thereof, or the like, of a user and/or patient 12. A patient is considered a person who desires to be treated for poison ivy, rashes, skin irritants, dermal inflammation, or the like. A patient may also be the user who is self-administering the composition. Furthermore, a medical professional 14 or the like may also be involved who is injecting via a needle, administering an oral dosage of a tablet 20, applying a skin patch 24, applying a skin cream 22, or the like.

### Communication System:

In the arrangement shown, as one example, the system and method 10 may also include a communication system 16. Communication system 16 may be a variety of different types of systems. This includes, but is not limited to a communication system 16 which a medical professional and patient use to communicate needs, dosages, diagnosis and the like. Similarly, a communication system 16 may be a biosensor which communicates metrics of the body to a user. Similarly, the biomedical communication system may be a system which communicates metrics to a medical professional and/or a user.

### Composition:

In the arrangement shown, as one example, the composition is for a dose of lysine 110. A dose of lysine may include lysine, inactive ingredients and/or compounds, including but not limited to, vitamins 111, sugars 112, vitamin B1 113, pill casings 114, Vitamin B2 115, gluten 116, Vitamin B6 117, omeprazole 118, Vitamin C 119, Iron 121, Glutamine 123, wheat products, other tablet formation features, chemical dyes, lactose, peanut oil, FODMAP sugars, and additional medication packages and formations. and the like. Various compositions are hereby contemplated for use as may be required for a users size, genetic makeup, allergens, a combination thereof, and the like.

Furthermore, and in the arrangement shown, as one example, the dose of lysine may include a skin patch 120. The skin patch is designed and configured to deliver transdermally a dosage. The dosage 122 may be delivered over time through slow release systems, and the like. Furthermore, the skin patch may also include an adhesive 124 for adhering the patch to the skin of a user in a comfortable manner and for a comfortable duration of time. In this way, the adhesive may be applied to the skin on various locations of a user such as the stomach, back, chest, arm, forearm, leg, foot, and the like.

One or more applications and/or methods are contemplated for use. Furthermore, the injection may also be a patch which injects through a plurality of pictures in a dermal surface.

A composition containing lysine for the treatment of poison ivy, a composition containing lysine for the prevention of poison ivy, processes, and methods of use are presented. Said another way, the present disclosure provides a composition and treatment including Lysine for the treatment and/or prevention of urushiol-induced contact dermatitis (otherwise known as poison ivy and/or poison oak). The present disclosure relates to a composition containing lysine for the treatment and/or prevention of effects caused by exposure to urushiol and/or a liquid composed of plant sap. Furthermore, the present disclosure relates to a supplement which can be taken daily and/or as a treatment for poison ivy and/or other allergens and/or allergic reactions.

Said another way, the present disclosure provides for administering and/or ingesting orally dosages of lysine at particular times, intervals, and amounts for the prevention of poison ivy and the side effects, the treatment of poison ivy and/or side effects, and/or the combination of prevention and treatment of poison ivy and/or the side effects therefrom.

Variations in dosage, timing, and composition are disclosed herein. For the purpose of the present disclosure, the composition or substances of the composition may vary but should be understood to mean the substances of the present composition. Additionally, some examples are disclosed herein with particular dosages but are shown only for example. dosages may vary from user to user and various dosages, compositions, and substances are hereby contemplated for use.

Various substances may be included in the composition. In one example, various substances include, but are not limited to, lysine, zine, selenium, vitamin B2, vitamin B6, vitamin B12, vitamin C, vitamin D3, folic acid and/or bioflavonoids, inorganic salts, organic salts, zinc chloride, zinc oxide, zinc sulfate, zinc hydroxide, zinc acetate, zinc aspartate, zinc gluconate, zinc orotate, zinc lactate, a combination thereof, and the like.

Furthermore, in the arrangement shown, as one example, lysine is discussed in various terms as an essential amino acid. The term is used for simplicity herein as “lysine” but is not meant to by limiting and is considered synonymous with “L-lysine” and/or L-lysine as an endogenous amino acid, and the like.

Dosages and range. In the arrangement shown herein, and in various examples, particular doses of lysine are discussed for simplicity. However, these dosages and amounts within the composition may vary and are hereby contemplated for use. This may include but is not limited to greater than or equal to 0.01 grams to less than or equal to 10 grams, greater than or equal to 0.1 grams to less than or equal to 9 grams, greater than or equal to 1 grams to less than or equal to 8 grams, greater than or equal to 2 grams to less than or equal to 7 grams, greater than or equal to 3 grams to less than or equal to 6 grams, and the like.

Furthermore, the amount of times dosages are taken per day, week or month may also vary depending on the user and a number of other factors as are hereby contemplated for use. As an example, dosages may be 1 time per month, 1 time per week, 1 time per day, 2 times per day, 3 times per day, 4 times per day, 5 times per day, 6 times per day, 7 times per day, and the like.

Surprisingly, in the state of the art, the dosages described herein are effective and have been shown effective in suppressing and/or reducing, and/or eliminating the reactions caused by urushiol and/or poison ivy exposure. The reactions of itchiness, redness, swelling, blisters, irritation, and the like are all reactions of exposure. Other reactions and reductions from the dosages described herein are to hives, bumps, redness, and more. These dosages of the composition are effective as a treatment, prevention, suppressive function, a combination thereof, and the like.

In the arrangement shown, as one example, the composition is configured in pill form. However, other composition formats are hereby contemplated for use. Other composition forms include, but are not limited to, solids, gels, chewing tablets, chewable coated gel, chewable coated pill, lozenge, pastille, orally disintegrating tablet, gel, metered dose spray, and/or chewing gum.

Administration for Prevention and Treatment: This example is not meant to be limiting but instead provide an example of administration of an example of the composition.

In one example, as an active prevention of reaction to poison ivy, three grams of lysine are consumed four to six times per day (or, in this example and said another way, 1.5 grams of lysine per 100 pounds of body weight), to prevent reactions when exposure to poison ivy and/or poison oak and/or urushiol has occurred. In this way, by dumping lysine into the system upon and/or shortly after exposure, the reaction can be prevented and/or reduced and/or greatly reduced through the increased presence of lysine and products made by lysine in the user's system.

In another example, for the pre-prevention of reactions to poison ivy, three grams are consumed two to three times per day for a month or two months prior to spring and/or outdoor seasons. In this way, by building up lysine in the system of a user and having lysine present and/or the building blocks created by lysine, reactions to poison ivy are prevented and/or less likely. Similarly, in this way, reactions to poison ivy can be prevented altogether in many different users.

In yet another example, when both pre-prevention of reactions to poison ivy and an active prevention dosage is taken, the active dosage (a dosage taken upon exposure or expected exposure) can be reduced from four to six times per day to two to three times per day. In this way, the lysine and other components are built up in the system prior to exposure so as to provide a lessened amount needed to introduce to the system of a user after exposure.

### L-Lysine Mitigation and Application:

In the arrangement shown, as one example, one method of biological pathways in which L-Lysine mitigates the effects of urushiol exposure on the body and/or dermatological layer of a user includes competitive inhibition in cellular uptake. In this way, the mechanisms of pathway mitigation is inhibition of the cellular uptake of arginine, which influences processes related to inflammation and immune response. In this way, L-Lysine, as disclosed herein, impacts the signaling pathways involved in the bodys' response to urushiol.

In the arrangement shown, as one example, one method of biological pathways in which L-Lysine mitigates the effects of urushiol exposure on the body and/or dermatological layer of a user includes immune modulation. In this way, L-Lysine as disclosed herein, modulates the immune responses of a user by influencing the production of inflammatory cytokines or other immune signaling molecules. Furthermore, in this way, the modulation affects the intensity and duration of the immune response triggered by exposure to urushiol.

In the arrangement shown, as one example, one method of biological pathways in which L-Lysine mitigates the effects of urushiol exposure on the body and/or dermatological layer of a user includes modulation of immune cell activation. In this way, L-Lysine, as disclosed herein, influences immune cell activation and extends to the recruitment and activation of immune cells at the site of urushiol exposure. Furthermore, in this way, the modulation results in a more controlled and aggressive immune response, mitigating the formation of blisters and more intense reactions to urushiol, as described further herein.

In the arrangement shown, as one example, one method of biological pathways in which L-Lysine mitigates the effects of urushiol exposure on the body and/or dermatological layer of a user includes inhibition of inflammatory mediators. In this way, L-Lysine, as disclosed herein, interferes with the production and/or release of inflammatory mediators, such as but not limited to, prostaglandins and leukotrienes. In this way, this modulation plays a role in vasodilation and increased vascular permeability, and/or processes that contribute to the formation of reactions. In this way, by modulating these mediators, L-Lysine doses and administration as disclosed herein, attenuate the effects and/or reactions to urushiol.

In the arrangement shown, as one example, one method of biological pathways in which L-Lysine mitigates the effects of urushiol exposure on the body and/or dermatological layer of a user includes collagen synthesis and tissue repair and/or tissue regeneration. In this way, L-Lysine, as disclosed herein, engages collagen synthesis and/or skin structure for tissue repair, reducing the severity of inflammation, blistering, and the like as caused by immune system reactions and/or caused by urushiol exposure.

In the arrangement shown, as one example, one method of biological pathways in which L-Lysine mitigates the effects of urushiol exposure on the body and/or dermatological layer of a user includes antiviral-like effects. In this way, L-Lysine, as disclosed herein, and the antiviral properties of L-Lysine impact viral infections beyond poison ivy as associated with various types of infections. In this way, L-Lysine, as disclosed herein interferes with and/or reduces viral replication and/or cellular entry mechanisms associated with viral impacts.

In the arrangement shown, as one example, one method of biological pathways in which L-Lysine mitigates the effects of urushiol exposure on the body and/or dermatological layer of a user includes antioxidant defense. In this way, L-Lysine, as disclosed herein, also applies beyond poison ivy to other ailments such as herpes infections and the like. Said another way, L-Lysine, as disclosed herein, exhibits antioxidant properties which counteract oxidative stress, protecting cellular structures and reducing the severity of skin reactions.

In the arrangement shown, as one example, one method of biological pathways in which L-Lysine mitigates the effects of urushiol exposure on the body and/or dermatological layer of a user includes microbiome modulation. In this way, L-Lysine, as disclosed herein, includes gut microbiome transformations and/or interference which affects systemic immune response. In this way, the L-Lysine, as disclosed herein, causes changes in the gut microbiome of a user which impact immune function. Said another way, these alterations impact the user's response to urushiol.

In the arrangement shown, as one example, one method of biological pathways in which L-Lysine mitigates the effects of urushiol exposure on the body and/or dermatological layer of a user includes neurotransmitter modulation. In this way, L-Lysine, as disclosed herein, causes changes to neurotransmission. In this way, L-Lysine, as disclosed herein influences communication between nerve cells and immune cells and/or immune system responses, which impact the users response to urushiol.

In the arrangement shown, as one example, one method of biological pathways in which L-Lysine mitigates the effects of urushiol exposure on the body and/or dermatological layer of a user includes neurotransmitter-mediated effects. In this way, L-Lysine, as disclosed herein, L-Lysine as a precursor for serotonin, impacts nerve cell communication in the skin. In this way, the users skin is impacted and the user is more comfortable because those influences which cause itchiness and/or discomfort after exposure are reduced and/or eliminated. Furthermore, in this way, damage to the skin and the like from itching and/or discomfort is reduced, further reducing discomfort in what is a common cycle of skin irritation and the like after exposure.

In the arrangement shown, as one example, one method of biological pathways in which L-Lysine mitigates the effects of urushiol exposure on the body and/or dermatological layer of a user includes epigenetic regulation of inflammatory gene expression. In this way, L-Lysine, as disclosed herein, influences the expression of genes involved in inflammatory responses. In this way, modulating gene expression regulates the intensity and duration of the inflammatory response to urushiol.

### Administration for Treatment:

This example is not meant to be limiting but instead provide an example of administration of an example of the composition.

In one example, as an active prevention of reaction to poison ivy, three grams of lysine are consumed four to six times per day to prevent reactions when exposure to poison ivy and/or poison oak and/or urushiol has occurred. In this way, by dumping lysine into the system upon and/or shortly after exposure, the reaction can be prevented and/or reduced and/or greatly reduced through the increased presence of lysine and products made by lysine in the user's system.

Administration for Prevention: This example is not meant to be limiting but instead provide an example of administration of an example of the composition.

In another example, for the pre-prevention of reactions to poison ivy, three grams are consumed two to three times per day for a month or two months prior to spring and/or outdoor seasons. In this way, by building up lysine in the system of a user and having lysine present and/or the building blocks created by lysine, reactions to poison ivy are prevented and/or less likely. Similarly, in this way, reactions to poison ivy can be prevented altogether in many different users.

Administration for Combination of Prevention and Treatment: This example is not meant to be limiting but instead provide an example of administration of an example of the composition.

In yet another example, when both pre-prevention of reactions to poison ivy and an active prevention dosage is taken, the active dosage (a dosage taken upon exposure or expected exposure) can be reduced from four to six times per day to two to three times per day. In this way, the lysine and other components are built up in the system prior to exposure so as to provide a lessened amount needed to introduce to the system of a user after exposure.

In one example, and only presenting an example of dosage, a 220 pound male may desire 3 grams of lysine 4 to 6 times per day as enough quantity and intake to eliminate and/or reduce skin irritations in circumstances where exposure and/or intense exposure have occurred. In another example, a user may desire to take 3 to 6 grams a day for a month or two months prior to being outside and/or hiking, and/or other circumstances where exposure is possible so as to prevent skin irritation and the like.

In another example, and only presenting an example of dosage, a user may desire to take 3 to 6 grams a day for a month or two months prior to being outside and/or hiking, and/or other circumstances where exposure is possible so as to prevent skin irritation and the like. Said another way, a user may desire to take the dosage for a duration of time when spring is approaching knowing that urushiol will be released by plants at certain times of the year.

In another example, and only presenting an example of dosage, a user may desire to take 3 to 6 grams a day for a month or two months prior to being outside and/or hiking, and/or other circumstances where exposure is possible so as to prevent skin irritation and the like. Said another way, a user may desire to take the dosage for a duration of time when spring is approaching knowing that allergens and other irritants will be in the air and/or that plants will be active and/or environmental conditions will cause active conditions for skin irritation.

In another example, and only presenting an example of dosage, a user may desire to take 3 to 6 grams a day for a month or two months prior to entering a particular job site and/or working condition in which a condition has a known irritant. Said another way, a user may desire to take the dosage for a duration of time when spring is approaching knowing that a particular irritant to the inflammation system of a user may be present, causing discomfort, or the like.

Some other conditions, environments, and/or examples included but are not limited to, herpes complex (given some similarities to a user's neurotransmitter system, and immunological responses, and cellular responses), poison ivy and/or poison oak, acute and/or chronic urticaria and/or hives, postherpetic neuralgia and/or shingles, construction sites with sawdust, chlorofluorocarbons, and/or other aspects of irritants.

In addition to the above identified features, options, controls, and components, compositions may also include other features and functionalities, among other options, controls, and components.

It will be appreciated by those skilled in the art that other various modifications could be made to the compositions, processes, and methods of use without parting from the spirit and scope of this disclosure. All such modifications and changes fall within the scope of the claims and are intended to be covered thereby.

What is claimed:

1. A method for the treatment of dermatological reactions to poison ivy, comprising:
injecting a dose of lysine.

2. The method of claim 1, further comprising:
wherein injecting a dose of lysine is by oral consumption; the oral consumption of the dose of lysine being consumption of a tablet;
wherein the tablet includes inactive compounds which help stabilize the dose of lysine;
wherein the tablet includes inactive compounds which aid in absorption of the dose of lysine.

3. The method of claim 1, further comprising:
wherein injecting a dose of lysine is by dermatological consumption; the dermatological consumption of the dose of lysine being a skin patch.

4. The method of claim 1, further comprising:
wherein injecting a dose of lysine is by dermatological consumption; the dermatological consumption of the dose of lysine being a cream applied to a skin surface.

5. The method of claim 1, further comprising:
wherein injecting a dose of lysine is by oral consumption; the oral consumption of the dose of lysine being consumption of a tablet;
wherein the tablet is consumed 1 to 2 times per day.

6. The method of claim 1, further comprising:
wherein injecting a dose of lysine is by oral consumption; the oral consumption of the dose of lysine being consumption of a tablet;
wherein the tablet is consumed 3 to 6 times per day.

7. The method of claim 1, further comprising:
wherein injecting a dose of lysine is by oral consumption; the oral consumption of the dose of lysine being consumption of a tablet;
wherein the tablet is consumed 1 to 2 times per day;
wherein the tablet is formed of 1 to 5 grams of lysine.

8. The method of claim 1, further comprising:
wherein injecting a dose of lysine is by oral consumption; the oral consumption of the dose of lysine being consumption of a tablet;
wherein the tablet is consumed 3 to 6 times per day;
wherein the tablet is formed of 2 to 3 grams of lysine.

9. The method of claim 1, further comprising:
wherein injecting a dose of lysine is by oral consumption; the oral consumption of the dose of lysine being consumption of a tablet;
wherein the tablet is consumed 1 to 10 times per day;
wherein the tablet is formed of 0.1 to 10 grams of lysine.

10. The method of claim 1, further comprising:
wherein injecting a dose of lysine is incrementally increased;
wherein incrementally increasing the dose of lysine occurs over a duration of time with lower dosage amounts during early dosage periods.

11. The method of claim 1, further comprising:
wherein injecting a dose of lysine is incrementally increased;
wherein incrementally increasing the dose of lysine occurs over a duration of time with lower dosage amounts during early dosage periods;
wherein incrementally increasing the dose of lysine occurs over the duration of time with lower dosage applications during early dosage periods.

12. A method for the prevention of dermatological reactions, comprising:
injecting a dose of lysine;
injecting the dose of lysine for a duration of time prior to exposure to poison ivy.

13. The method of claim 12, further comprising:
wherein the dermatological reaction is inflammation from exposure to poison ivy.

14. The method of claim 12, further comprising:
wherein the dermatological reaction is shingles.

15. The method of claim 12, further comprising:
wherein the dose of lysine is formed of a nutraceutical amino acid complex.

16. The method of claim 12, further comprising:
injecting the dose of lysine approximately 1 day to 52 weeks prior to exposure.

17. The method of claim 12, further comprising:
injecting the dose of lysine approximately 1 day to 12 weeks prior to exposure.

18. The method of claim 12, further comprising:
injecting the dose of lysine approximately 1 week to 8 weeks prior to exposure.

19. The method of claim 12, further comprising:
injecting the dose of lysine approximately 2 weeks to 4 weeks prior to exposure.

20. A method for the treatment and prevention of reactions to urushiol-induced contact dermatitis, comprising:
injecting a dose of lysine; wherein the dose of lysine includes L-Lysine;
wherein injecting a dose of lysine refers to introducing and/or causing lysine to absorb into the system of a user;
decreasing a subsequent amount of the dose of lysine over a duration of time after an initial dose of lysine cycle is completed.
